(19) United States
(12) Patent Application Publication
(10) Pub. No.: US 2005/0171214 A1
(43) Pub. Date: Aug. 4, 2005

(54) Medicament for improving the failure of accommodation

(75) Inventors: Seiji Hayasaka, Toyama (JP); Yasunori Nagaki, Toyama (JP); Takatoshi Uonomi, Toyama (JP); Mari Sanada, Toyama (JP)

Correspondence Address: FLYNN THIEL BOUTELL & TANIS, P.C., 2026 RAMBLING ROAD, KALAMAZOO, MI, 49008-1699, US

(73) Assignee: Fuji Chemical Industry Co., Ltd.

(21) Appl. No.: 11/098,152

(22) Filed: Apr. 4, 2005

Related U.S. Application Data
(62) Division of application No. 10/477,624, filed on Nov. 12, 2003.

(30) Foreign Application Priority Data
May 24, 2001 (JP) 2001-155575

(51) Int. Cl. A61K 31/225; A61K 31/12; A61K 31/07

(52) U.S. Cl. 514/690; 514/725; 514/548

(57) ABSTRACT

There are disclosed in this specification a useful medicament for therapy and/or prevention of the failure of accommodation and a food and drink having an improving effect against failure of accommodation. The present invention relates to a medicament for improving the failure of accommodation which contains astaxanthin and/or its esters, and to a food and drink causing an improving effect against the failure of accommodation which contains astaxanthin and/or its esters.

### Technical Field

The present invention relates to a medicament for improving the failure of accommodation comprising astaxanthin and/or its esters, and to a food and drink causing an improving effect against the failure of accommodation comprising astaxanthin and/or its esters.

### Background Art

The human eyes automatically accommodate so as to focus the retina by thickening the lens for near point vision or otherwise by thinning them for far point vision. The failure of this accommodation includes presbyopia which shows a difficulty in near point accommodation caused by poor accommodability due to age-deterioration, and morbid abnormalities of accommodation such as weakness of accommodation, hypocyclosis, dullness of accommodation, accommodation paralysis, tonic accommodation, accommodation spasm, etc. More specifically, causes of the latter disorders may include eye-fatigues such as ciliary fatigue, fatigue of the ocular muscle which moves the eyeball and fatigue of the optic nerves, and systemic diseases or other ophthalmic diseases. In the treatment of these disorders, it is said that no therapeutic method exists for the presbyopia, which may only be corrected symptomatically with glasses or contact lens in order to improve poor accommodability. For the morbid abnormalities of accommodation, therapy of the underlying disease or environmental improvement may be performed. Their symptomatic treatment may include correction with glasses or doses of vitamin B.

Consequently, it is an actual situation that there are very few therapeutic methods for failures of accommodation, especially, there is almost no preventive method for them. A method of using astaxanthin and/or its esters for therapy of retinal damage or retinal diseases is reported (U.S. Pat. No. 5,527,533 specification), and it is reported that food and drink comprising astaxanthin and/or its esters are permissible as an edible composition which have a preventive effect against cataracts or an inhibiting effect against their progression can inhibit the crisis or progression of cataracts, and furthermore, can inhibit monocular diplopia, asthenopia or halation complicated with the disorder of visual acuity associated with cataracts (JP 10-276721 A)). However, there is no report on medicaments for improving the failure of accommodation comprising astaxanthin and/or its esters, or on a food and drink having an improving effect against the failure of accommodation comprising astaxanthin and/or its esters.

### Disclosure of the Invention

An object of the present invention is to provide a useful medicament for therapy and/or prevention of the failure of accommodation and further to provide a food and drink having an improving effect against the failure of accommodation.

As a result of having searched effective compounds for improving the failure of accommodation, the present inventors have found that astaxanthin and/or its esters are useful as a medicament for improving the failure of accommodation, and also found that a food and drink containing astaxanthin and/or its esters as a component thereof show an improving effect against the failure of accommodation. Thus, the invention has made based on the above findings.

That is, the invention is a medicament for improving the failure of accommodation which comprises astaxanthin and/or its esters, and a food and drink having an improving effect against the failure of accommodation which contain astaxanthin and/or its esters.

Astaxanthin and/or its esters used as the effective ingredients in the present invention may be chemically synthesized or extracts or crude extracts derived from natural origin. Those may be used singly or in a suitably mixed form. Examples of those derived from natural origin include crusts, eggs and organs of crustaceans such as shrimp, krill, crab and the like; skins and eggs of various fishes and shellfishes; algae such as Haematococcus, etc.; yeasts such as Phaffia red yeast, etc.; oceanic bacteria such as Agrobacterium auranticum; and seed plants such as Adonis amurensis and Ranunculus acris. Naturally extracted products and synthesized products are put on the marketplace and hence they are easily available.

Astaxanthin and/or its esters can be obtained by cultivation of e.g. Phaffia red yeast, Haematococcus green algae, Agrobacterium auranticum, etc., in an appropriate medium in accordance with the conventional methods or the known methods.

Various methods are known for the extraction of astaxanthin from the above cultivated substances or for extraction and purification from the above crustaceans. For example, since the diester form of astaxanthin has a liposoluble property, astaxanthin components can be extracted from natural sources containing astaxanthin with liposoluble organic solvents such as acetone, alcohol, ethyl acetate, benzene, chloroform, etc. After the extraction, the concentrated diester form of astaxanthin can be obtained by removing the solvents according to a usual method. The concentrated astaxanthin diester can be further purified, if necessary.

Astaxanthin comprises 3,3′-dihydroxy-β,β-carotene-4,4′-dione and its stereoisomers. More specifically, three stereoisomers are known as (3R, 3′R)-astaxanthin, (3R, 3′S)-astaxanthin and (3S, 3′S)-astaxanthin. Any of them can be used in the present invention.

It is known that astaxanthin and/or its esters have not been observed having any mutagenicity but are highly safe compounds.

As the astaxanthin component in the present invention, any of the free form, monoester and diester forms may be used. The diester form is more stable physically than the free or monoester form and hard to subject to oxidative decomposition, because its two hydroxy groups are protected by ester bonds. However, when it is taken into the living body, it is considered to quickly hydrolyze into free astaxanthin by bioenzymes to exert its effect.

Monoesters of astaxanthin include lower or higher saturated fatty acid esters, or lower or higher unsaturated fatty acid esters. Specifically, the monoesters include the ester forms of acetic acid, lauric acid, myristic acid, pentadecanoic acid, palmitic acid, palmitoleic acid, heptadecanoic acid, elaidic acid, ricinoleic acid, petroselinic acid, vaccenic acid, eleostearic acid, punicinic acid, licanoic acid, palynalic acid, gadolic acid, 5-eicosenoic acid, 5-docosenoic acid, cetolic acid, ercinoic acid, 5,13-docosadienoic acid, selacholic acid, decenoic acid, stering acid, dodecenoic acid, oleic acid, stearic acid, eicosapentaenoic acid, docosahexaenoic acid, linoleic acid, linolenic acid, arachidonic acid, etc.

Diesters of astaxanthin include diesters composed of the same or different fatty acids selected from the above fatty acids.

Furthermore, examples of astaxanthin esters include esters of an amino acid such as glycine, alanine or the like; esters of a mono- or poly-carboxylic acid and their salts such as citric acid esters, etc.; or inorganic acid esters and their salts such as phosphoric acid esters, sulfuric acid esters, etc.; glyco-esters such as glucoside, etc.; mono esters such as glyco-fatty acid esters, glycoglycero-fatty acid esters, sphingoglyco-fatty acid esters, glycero-fatty acid esters, glycero-phosphoric acid esters, etc. Or they include the same or different diesters selected from the above amino acids, carboxylic acids, phosphoric acids, sulfuric acids, sugars, unsaturated fatty acids, saturated fatty acids, polyunsaturated fatty acids, fatty acid esters, glyco-fatty acid esters, glycoglycero-fatty acid esters, sphingoglyco-fatty acid esters, glycero-fatty acid esters, glycero-phosphoric acid esters, etc.

The medicament of the present invention which is a medicament for improving the failure of accommodation comprising astaxanthin and/or its esters can be prepared in various dosing forms according to the conventional methods, by appropriately combining with sugars such as lactose, saccharose, etc., amino acids such as glycine, etc., excipients such as cellulose, etc., binders such as starch, gelatin, methyl cellulose, polyvinylpyrrolidone, etc., disintegrators such as starch, agar, etc., or lubricants such as silicon dioxide, talc, magnesium stearate, polyethylene glycol, etc., flavors and sweetening agents. For example, the medicament will be administered in such dosing forms as, solid forms such as tablets, powders, granules, fine granules, pills, enteric coated forms, capsules, troche, etc., oral liquid preparations such as elixirs, syrups, etc., liquid forms such as suspensions, emulsions, syrups, external liquid preparations, fomentations, nasal drops, ear drops, eye drops, etc., or capsules filled with an oil or fat such as soft capsules, etc., inhalants, lotions, suppositories, enteral nutrients, etc.

Astaxanthin and/or its esters are easily oxidized by ambient oxygen and unstable against temperature or light. They show a tendency to decompose with the passage of time during storage in their preparation. In order to avoid this decomposition, antioxidants, as stabilizers, can be added to the above components, if necessary. For example, one or two or more mixtures selected from existing antioxidants such as, e.g., vitamin A, vitamin B, vitamin C and vitamin E (tocopherol and tocotrienol) or their derivatives, cysteine, glutathione, phytic acid, catechins, flavonoids, β-carotene, glutathione peroxidase, citric acids, phosphoric acids, polyphenols, nucleic acids, herb medicines, marine algae and inorganic substances can be added to the above components. It is desirable to dose them in a fine powder form or non-crystalline powder in order to increase the absorbability of free or monoester astaxanthin.

Although the dosage of astaxanthin and/or its esters to be used as the medicament may vary according to the age, body weight or grade of symptoms of a patient who receives the medicament, or its dosing form, the dose in terms of free astaxanthin will be in the range for adults for oral administration per day : 0.1 mg-10 g, preferably 0.1 mg-1 g and preventively in an amount of from 0.1 mg-100 mg, and in parenteral administration per day : 0.01 mg-1 g, preferably 0.01 mg-100 mg and preventively in an amount of from 0.01 mg-10 mg.

Although the time to be administered is not limited particularly, it is recommended to administer astaxanthin and/or its esters preferably during hunger or 30 minutes before a meal for good efficiency.

As the medicament of the present invention increases the human eye-accommodability, it is useful as a preventive and/or therapeutic agent for a condition of causing the failure of eye-accommodability such as, i.e., presbyopia caused by difficulty in near point accommodation caused by poor accommodability due to age-deterioration, weary eyes of a person who works in a VDT operation or works in operations which overload the eye, or patients with such morbid abnormalities as weakness of accommodation, hypocyclosis, dullness of accommodation, accommodation paralysis, tonic accommodation, accommodation spasm, etc.

Incidentally, even though the content disclosed in U.S. Pat. No. 5,527,533 specification is directed to “eye”, it is restricted to the retina and its linked nerve. Also, JP 10-276721 A merely discloses a cataract and its causing asthenopia. On the other hand, the medicament for improving failure of accommodation involving in the present invention is considered that morbid abnormalities of accommodation may be ameliorated by amelioration in the bloodstream to the ciliary body and preventing injuries to the ciliary body muscle and by the control nerve (parasympathetic).

The present invention relates also to a food and drink having an improving effect against a failure of accommodation which comprises astaxanthin and/or its esters.

The food and drink to which astaxanthin and/or its esters are added include such general foods as, e.g. margarine, butter, butter sauce, cheese, raw cream, shortening, lard, ice cream, yogurt, diary products, meat sauce products, fish products, fried potato, potato chips, popcorn, a seasoned powder for sprinkling over rice, chewing gum, chocolate, pudding, jelly, gumi-candy, candy, drops, caramel, sponge cake, cake, doughnut, biscuit, cookie, cracker, etc., macaroni, pasta, salad oils, instant soup, dressing, egg, mayonnaise, miso., etc., or carbonated or non-carbonated drinks such as fruit drinks, refreshing drinks, sports drinks, etc., non-alcoholic drinks such as tea, coffee, cocoa, etc., or liquors such as liqueur, medical liquor, etc.

The food and drink of the present invention can be processed by usual methods, combining astaxanthin and/or its esters with raw materials of the general foods. Although the combining quantity of astaxanthin and/or its esters may vary depending on the food form and so on, generally, it is desirable that the combining quantity as free astaxanthin lies in a range of 0.1 mg-10 g, preferably 1 mg-1 g and preventively 0.1 mg-100 mg. For foods and drinks, functional foods and nutritional supplements, the combining quantity will be adjusted in preparations with the necessary quantity to exert the improving effect against the failure of accommodation. The quantity for usage can be selected appropriately depending on the kind of food and drink by persons having an ordinary skill in the art.

When the food and drink of the present invention are used as nutritional and supplemental foods or functional foods, their forms may be the same as the above-described medicament forms. There may also be used a mixture of materials such as milk protein, soybean protein, egg albumin protein, etc., or their decomposed material such as albumin oligopeptide, soybean hydrolyzate and amino acid unit. The food can also be formed into natural liquid foods, semi-digested nutritional foods and nutritional foods, drinks, capsules or enteral nutrients, etc. combined with sugars, fats, trace elements, vitamins, emulsions, flavors, etc. For the drink form, the material can be combined with the drink as nutritional additives such as amino acids, vitamins, minerals, etc., and sweetening agents, spices, flavors, pigments, etc., in order to keep a balance in the components or to impart a good taste for consumption. Furthermore, such natural extracts as blueberry extract, etc., containing a large amount of anthocyanin, which may be good for the eye, may be added, thereby a synergistic effect may be exerted. The form of the food, etc. in the present invention is not limited thereto.

### THE BEST MODE FOR CARRYING OUT THE INVENTION

The following Examples and Preparation Examples illustrate the present invention in details but the present invention is not restricted thereto.

### EXAMPLE 1:

### Improving Effect of Astaxanthin on Eye-Accommodability

(Test method)

People satisfying the following selection standards were used as subjects.

(1) A person having subjective symptoms of eyestrain or working for VDT operation, (2) 1.0 or more of both eyes in vision after correction. (3) 35-59 years old, (4) persons who do notusually take any medicines or health foods, (5) a person who can keep in compliance with all the test-related requirements and take the medical examinations stipulated by the test method.

Persons who have retinal disorders or cataracts were excluded from the subjects.

A test food with 5 mg /capsule of astaxanthin and a control food with 0 mg /capsule of astaxanthin were prepared. The test was conducted in a double-blind method.

I. Before Intake

After completion of the accommodability test for the subjects using an accommodometer by which the change in the refraction value (accommodation reaction) during moving object can be measured continuously and objectively, and accommodation abnormalities including VDT syndrome can be detected clearly.

A person in charge of the test prepared a subjects' name-list stratified by sex and the test results and handed it to a controller. The controller prepared an allocation table separating the subjects into test food group and control food group based on the name list. In addition, the controller stuck each label with a subject's name on the test food or control food according to the allocation table. The controller sealed up the allocation table.

II. During Intake

Each of the subjects took one capsule a day after supper continuously for 4 weeks.

III. After Completion of Intake

Each of the subjects took the accommodation examination by the accommodometer. The results are shown in Table 1 which indicates the human eye-accommodabilites in the test food group and the control group.

Incidentally, the value of accommodability (dioptres) in Table 1 is represented in mean ± standard deviation and “★” in Table 1 means significant difference p<0.01, before intake vs. after intake (t-test).

TABLE 1

| | | | Accommodability |
|---|---|---|---|
| | | Number | (Dioptres) |
| Test food Group | Before Intake | 26 | 2.279 ± 1.442 |
| | After Intake | 26 | 2.775 ± 1.563* |
| Control food Group | Before Intake | 30 | 2.551 ± 1.744 |
| | After Intake | 30 | 2.728 ± 1.974 |

It was recognized from the results shown in Table 1 that when the human's eye-accommodability was compared between before intake of astaxanthin and after intake of astaxanthin for 4 consecutive weeks, it was increased in the test food group by a statistically significant difference. In contrast thereto, this difference was not recognized in the control group. It can be understood that astaxanthin improves the eye-accommodability.

### EXAMPLE 2

The effects of astaxanthin on eye-accommodability, critical flicker fusion (CFF) and pattern visual evoked potentials (PVEP) were evaluated in more detail.

As a control, 13 persons who have not been administered astaxanthin and have not worked in a VDT operation were indicated as the A group. 26 VDT workers were divided into two groups at random. The B group (13 persons) was orally administered 5 mg of astaxanthin/day for 4 consecutive weeks while the C group (13 persons) was orally administered a placebo for 4 consecutive weeks. No significant difference was recognized in age among the three groups.

A double-blind test was conducted for the B and C groups.

The eye-accommodability of the A group was 3.7±1.5 dioptres. Each eye-accommodability of the B and C groups before administration was 2.3+1.4 dioptres and 2.2+1.0 dioptres, respectively and significantly (p<0.05) lower than that of the A group.

The eye-accommodability of 2.8±1.6 dioptres in the B group after administration of astaxanthin became significantly (p<0.01) greater than that before the administration of the astaxanthin. On the other hand, the eye-accommodability (2.3+1.1 dioptres) in the C group after the administration of the placebo did not (appreciably) change.

With respect to eye-accommodability, the following values of normal persons by ages are known:

8 Years of age −13.8 dioptres, 16 years of age −12.0 dioptres, 24 years of age −10.2 dioptres, 32 years of age −8.2 dioptres, 40 years of age −5.8 dioptres, 48 years of age −2.5 dioptres, 56 years of age −1.25 dioptres, 64 years of age −1.1 dioptres (“Stedman's Medical Dictionary” the fourth edition, p. 615)

The critical flicker fusion, amplitude and latency of P100 in the pattern visual evoked potentials in the A group were 45±4.2 Hz, 6.5±1.8 μV, 101.3±6.5 msec, respectively.

The critical flicker fusion was significantly (p<0.01) lower in the B and C groups before administration than in A group.

The critical flicker fusion in the B and C groups did not (appreciably) change after administration. The amplitude and the latency of P100 in the pattern visual evoked potentials in the B and C groups before the administration were the same as with those in the A group. They did not appreciably change after administration.

It is suggested from the findings of this study that the eye-accommodability of the VDT workers may be improved after the administration of astaxanthin.

VDT operation is reported to induce various visual disorders including eyestrain, blurring and double vision (such a status that a single object is observed as two objects) and to have an adverse effect on the visual system.

The eye-accommodability, amplitude and prolonged latency in the pattern visual evoked potentials are used for determining the degree of eyestrain.

Subjects and method:

13 people who had not been engaged in a VDT operation were indicated as a healthy control group (A group). Most of them worked outdoors.

Also, 26 workers were selected who had been engaged in a VDT operation for 4 hours per day, for 5 days (Monday to Friday every week) per week and for a year or more. Their eyes were better than twenty-twenty (20/20). All of them wore eyeglasses for accommodation during the VDT operation.

Herein, persons who have worn contact lens, persons who have used eye drops within the past 6 months, persons who have suffered from heavy ocular disorders including diabetes mellitus and persons who have suffered from systemic diseases were excluded from the subjects.

A double-blind test was conducted with respect to the VDT workers. The VDT workers were divided into an astaxanthin-administration group (n=13, B group) and placebo-administration group (n=13, C group). There was no difference in age among the three groups (Table 2 shown below).

TABLE 2

| | |
|---|---|
| Non-VDT workers | VDT workers |

| | A group | B group | C group |
|---|---|---|---|
| Number of | 13 | 13 | 13 |
| Subject | | | |
| Male | 11 | 11 | 10 |
| Female | 2 | 2 | 3 |
| Average | 47.6 ± 4.5 | 47.8 ± 4.3 | 47.5 ± 4.8 |
| of age | | | |
| Range | 39-53 | 40-53 | 38-53 |
| of age | | | |

An astaxanthin capsule (5 mg/capsule) was orally administrated to each of the B group subjects one time a day 30 minutes before supper.

The astaxanthin was prepared from Haematococcus pluvialis extract (a product of Fuji Chemical Industry Co., Ltd.).

A placebo capsule was orally administrated to each of the C group subjects one time a day 30 minutes before supper.

The B and C group subjects did a usual VDT operation in the administration period. The A group subjects did not receive any administration. Measurement for eye-accommodability, critical flicker fusion and pattern visual evoked potentials:

All of these measurement items were conducted with each right eye of the subjects at a.m. 9:00-12:00 on a Saturday.

The eyesight was measured at each distance of 5 m and 35 cm using a Landolt ring.

The eye-accommodability was evaluated by measurement of the near and far points.

The near point was measured with a D'Acomo apparatus (binocular opening constant point refraction near point ruler, a product of World Optical Corporation) according to the Uozato et al' method (Uozato H, Nagakawa A, Hirai H, Saishin M: A new near-point ruler using constant dioptric stimulus. Folia Ophthalmol Jpn 1988;39:1247-1248).

The far point was measured in the best-corrected refraction for each of the subjects.

The eye-accommodability (dioptres) was calculated by subtracting the far point (dioptres) from the near point (dioptres).

The critical flicker fusion was determined by decreasing the frequency of the signals at a constant speed using a C.F.F. tester (a product of Yagami Co., Ltd.). There was used the average value of the three times value measured by the individual eyes of the subjects.

The pattern visual evoked potential was recorded according to the method established by the International Society for Clinical Electrophysiology of Vision to measure one positive peak strength (P100) and the latency (the difference in μV between N75 peak and P100 peak).

Statistical Analysis

The data on before and after administration were analyzed statistically using a paired t-test. Also, the data for the A and B groups and those for the A and C groups were measured in an unpaired test. The probability value is below 0.05 that is considered to be significant.

From the above results, no systemic side effects were recognized in the B and C groups.

The eyesight of the B and C subjects at both the distances of 5 m and 35 cm did not appreciably change before and after administration. Each numerical value of the eye accommodability, critical flicker fusion and pattern visual evoked potentials are shown in the table below.

Incidentally, in mean ± standard deviation in the table, the mark #(p<0.01) was compared with the value before administration, and the mark * (p<0.05) was compared with the value for the A group.

TABLE 3

| | | VDT worker | | | |
|---|---|---|---|---|---|
| | Non-VDT | B group | | C group | |
| | Workers | (n = 13 eye) | | (n = 13 eye) | |
| | A group | Before | After | Before | After |
| | (n = 13 eye) | Admini- | Admini- | Admini- | Admini- |
| | Not | stration | stration | stration | stration |
| | Admini- | of | of | of | of |
| | strated | astaxanthin | astaxanthin | placebo | placebo |
| Accommod- | 3.7 ± 1.5 | 2.3 ± 1.4* | 2.8 ± 1.6# | 2.2 ± 1.0* | 2.3 ± 1.1 |
| ability | | | | | |
| (D) | | | | | |
| CFF (Hz) | 45.0 ± 4.2 | 39.9 ± 5.3 | 38.4 ± 4.8 | 39.9 ± 5.5* | 38.4 ± 3.9 |
| PVEP-P100 | 6.5 ± 1.8 | 5.8 ± 1.7 | 5.6 ± 1.6 | 5.7 ± 2.3 | 5.5 ± 1.3 |
| (μV) | | | | | |
| Amplitude | | | | | |
| PVEP-P100 | 101.3 ± 6.5 | 102.5 ± 6.9 | 104.8 ± 7.4 | 104.4 ± 5.7 | 105.2 ± 5.7 |
| Latency | | | | | |
| (msec) | | | | | |

The eye-accommodability in the A group was 3.7±1.5 dioptres.

The eye-accommodabilities in the B and C groups before administration were 2.3±1.4 dioptres and 2.2±1.0 dioptres, respectively and significantly (p<0.05) lower than in the A group.

The eye-accommodability in the B group after administration was 2.8±1.6 dioptres, and thus became significantly (p<0.01) greater than before administration.

The eye-accommodability in the C group after administration of the placebo was 2.3±1.1 dioptres and thus it did not (appreciably) change.

The P100 strength in PVEP in the A group was 6.5±1.8 μV. The P100 strengths in the B and C groups before administration were 5.8±1.7 μV and 5.7±2.3 μV, respectively, each being substantially the same as that in the A group.

There was no significant difference in the P100 strength between the A and B groups. The strengths in the B and C groups after administration were respectively 5.6±1.6 μV and 5.5±1.3μV, each being substantially the same as that before administration.

The 100 latency in PVEP in the A group was 101.3±6.5 msec. The latencies in the B and C groups before administration were respectively 102.5±6.9 msec and 104.4±5.7 msec, each being substantially the same as that in the A group. There was no significant difference in the latency between the B and C groups. The latencies in the B and C groups after administration were respectively 104±7.4 msec and 105.2±5.7 msec, each being the same as that in the A group.

The eye accommodability may be varied depending on the age. Herein, the ages among the three groups were matched with one another. Also, diabetes mellitus is a dangerous factor which causes a decrease in eye-accommodability. Therefore, a diabetes mellitus patient was excluded from this test.

The results of this test show that the eye-accommodability of the VDT workers may be improved by the administration of astaxanthin.

It is reported by Murata et al that in the VDT workers, the near point increases and the eye-accommodability decreases (Murata K; Araki S; Kawakami N; Saito Y, Hino E: Central nervous system effects and visual fatigue in VDT workers. Int. Arch Occup Environ Health 1991, 63(2), p109-113), Murata K; Araki S; Yokoyama K; Yamashita K; Okamatsu T; Sakou S: Accumulation of VDT work-related visual fatigue assessed by visual evoked potential, near point distance and critical flicker fusion. Ind. Health 1996, 34(2), 61-69). The authors suggest that the chronic stress caused by use of the VDT induces the hypofunction of the ciliary body and decreases the eye-accommodability.

It is reported that in the VDT workers their critical flicker fusion is lowered, their amplitude is decreased and their latency of P100 in PVEP is prolonged.

In this test, a slight critical flicker fusion was seen in the VDT workers. However, the critical flicker fusion was significantly different between before and after the test. In addition, no appreciable decrease in the P100 strength in PVEP was seen in the VDT workers.

In this test, the administration of astaxanthin does not cause any effect toward the critical flicker fusion and the pattern visual evoked potential derived from the nervous system since there is no significant difference in these items between before and after the administration of astaxanthin. On the other hand, the eye-accommodability may be significantly improved by the administration of astaxanthin. This suggests that astaxanthin acts on the ciliary body of the eye. The ciliary body does an important action for focusing on an object by changing the thickness of the lens. It exerts such an action that it stretches in order to make the lens thick for near vision while it loosens for far vision.

### PREPARATION EXAMPLE 1 (TABLET)

The ingredients shown below were uniformly mixed together in the following compositional ratio (wt. %) to make tablets, each being 180 mg in weight.

| | |
|---|---|
| Astaxanthin | 5% |
| Lactose | 75% |
| Ground magnesium oxide | 20% |

### PREPARATION EXAMPLE 2 (CAPSULE)

Haematococcus extracted oil (containing 10 wt. % of astaxanthin) was filled in a soft capsule film consisting of the following components according to a usual method to make soft capsules, each being 100 mg in weight.

| | |
|---|---|
| Gelatin | 70% |
| Glycerin | 23% |
| Propyl p-hydroxybenzoate | 0.5% |
| Water | p.q |
| Total | 100% |

### PREPARATION EXAMPLE 3 (CAPSULE)

The above-described Haematococcus extracted oil and blueberry extract were filled in the above-described soft capsule film in 1:1 weight ratio according to a usual method to make soft capsules, each being 100 mg in weight.

### PREPARATION EXAMPLE 4 (DRINK)

The ingredients shown below were compounded together and water was added thereto according to a usual method to prepare a drink.

| | |
|---|---|
| Astaxanthin | 5 g |
| Liquid sugar | 4 kg |
| Sodium DL-tartrate | 1 g |
| Citric acid | 50 g |
| Vitamin C | 50 g |
| Vitamin E | 150 g |
| Cyclodextrin | 25 g |
| Potassium chloride | 5 g |
| Magnesium sulfate | 2 g |

### PREPARATION EXAMPLE 5 (NUTRIENT AND TONIC)

The ingredients shown below were compounded together and water was added thereto according to a usual method to prepare a solution.

| | |
|---|---|
| Astaxanthin ethyl ester | 5 g |
| Liquid sugar | 4 kg |
| Sodium DL-tartrate | 1 g |
| Citric acid | 50 g |
| Vitamin B₁ | 10 g |
| Vitamin B₂ | 10 g |
| Vitamin B₆ | 10 g |
| Vitamin B₁₂ | 10 g |
| Vitamin C | 50 g |
| Vitamin E | 150 g |
| Folic acid | 5 g |
| Nicotinic acid | 10 g |
| Cyclodextrin | 25 g |
| Potassium chloride | 5 |
| Magnesium sulfate | 2 g |

### INDUSTRIAL APPLICABILITY

By the present invention there is provided a medicament for improving the failure of eye-accommodation which comprises astaxanthin and/or its esters, and a food and drink having an improving effect against the failure of accommodation which comprises astaxanthin and/or its esters. As astaxanthin and/or its esters improve the human eye-accommodability, the medicament is useful as a preventive and/or therapeutic agent for the condition where the failure of eye-accommodability occur, such as the presbyopia which shows difficulty in near point accommodation caused by poor accommodability due to age-deterioration, a weary eye of a person who works in a VDT operation or works with operations which overload the eye, or patients with such morbid abnormalities as weakness of accommodation, hypocyclosis, dullness of accommodation, accommodation paralysis, tonic accommodation, accommodation spasm, etc.

1. A method of treating a failure of accommodation in a subject comprising the step of administering to said subject a pharmacologically effective amount of astaxanthin and/or an ester thereof.

2. The method of claim 1, wherein the astaxanthin and/or ester thereof is administered orally in a daily amount of 0.1 mg-10 g as free astaxanthin.

3. The method of claim 1, wherein the astaxanthin and/or ester thereof is administered orally in a daily amount of 0.1 -100 mg as free astaxanthin.

4. The method of claim 1, wherein the astaxanthin and/or ester thereof is administered parenterally in a daily amount as 0.01 mg-1 g of free astaxanthin.

5. The method of claim 1, wherein the astaxanthin and/or ester thereof is administered parenterally in a daily amount of 0.01 -10 mg as free astaxanthin.

6. The method of claim 1, wherein the failure of accommodation is caused by weary eyes of a person who works for VDT operation or works for operations with overloading eye.

7. The method of claim 1, wherein the failure of accommodation is caused by presbyopia.

8. The method of claim 1, wherein the astaxanthin and/or ester thereof is administered in a food or a drink.

9. A method for preventing a failure of accommodation in a subject comprising the step of administering to said subject a pharmacologically effective amount of astaxanthin and/or an ester thereof.

10. The method of claim 9, wherein the astaxanthin and/or ester thereof is administered orally in a daily amount of 0.1 -100 mg as free astaxanthin.

11. The method of claim 9, wherein the astaxanthin and/or ester thereof is administered parenterally in a daily amount of 0.01 -10 mg as free astaxanthin.
